(19) United States
(12) Patent Application Publication
(10) Pub. No.: US 2013/0150695 A1
(43) Pub. Date: Jun. 13, 2013

(54) Medical Implant and Medical Arrangement

(71) Applicant: Biotronik SE & Co. KG, Berlin (DE)

(72) Inventors: Sarah Biela, Berlin (DE); Olaf Skerl, Bad Doberan (DE)

(73) Assignee: BIOTRONIK SE & CO. KG, Berlin (DE)

(21) Appl. No.: 13/692,050

(22) Filed: Dec. 3, 2012

Related U.S. Application Data
(60) Provisional application No. 61/568,176, filed on Dec. 8, 2011.

(51) Int. Cl. A61B 5/042; A61B 17/3205; A61N 1/05; A61N 1/365; A61N 1/39

(52) U.S. Cl. CPC A61B 5/042; A61N 1/365; A61N 1/3987; A61N 1/0587; A61B 17/3205
USPC 600/374; 607/17; 607/7; 607/119; 606/167

(57) ABSTRACT

A medical implant comprising a transducer element which induces mechanical vibrations of the implant when electrically and/or magnetically controlled.

### CROSS-REFERENCE TO RELATED APPLICATIONS

This patent application claims the benefit of co-pending U.S. Provisional Patent Application No. 61/568,176, filed on Dec. 8, 2011, which is hereby incorporated by reference in its entirety.

### TECHNICAL FIELD

The present invention relates generally to a medical implant and a medical arrangement comprising such an implant. In addition to implantable electrode leads (also referred to in the following description as “electrode leads”) or sensor leads of the type used in cardiac pacemakers or implantable cardioverters, for example, the term “implant” also relates—within the scope of this patent application—to so-called “leadless pacers” (e.g., leadless cardiac pacemakers), of the type described in European Patent No. EP 1 714 670, for example, or “leadless sensors” which are sensors comprising a transmitter/receiver unit and which can be implanted into the vascular system of a living organism or subcutanteously.

### BACKGROUND

The explantation of adhered electrode leads presents great difficulties to a physician. Previously, an electrode lead has been “cut out” for explantation, e.g., using laser cutting or conventional knives. At the same time, a tensile force must be applied to the electrode lead, although it must not cause the lead to tear. Special systems for achieving this difficult objective have been developed, such as, for example, an electrode extraction system from the company VascoMed GmbH.

Various coatings (e.g., polymer-based) have also been known for a long time, which are intended to impede the adhesion of electrode leads or other medical implants in the body, or coatings that release active agents (e.g., drug-eluting coatings) which are intended to prevent adhesion via this release of active agent.

A coating of an implant is achieved, for example, by embedding a hydrophilic interface between the implant surface and the bodily fluid. As a result, inflammatory reactions of the surrounding tissue and adhesions are minimized. A plurality of natural, synthetic and semi-synthetic materials are currently in use for implant coatings. Naturally occurring materials containing, for example, alginate, chitosan, collagen, dextrane and hyaluronan, synthetic polymer materials as coating materials are, e.g., poly(lactic acid) and poly(lactic co-glycolic acid) (PLGA), 2-hydroxyethyl methacrylate, poly(ethylene glycol) (PEG), and poly(vinyl alcohol) (PVA). However, not all of these materials exhibit the desired properties over the long term. Many do not provide adequate mechanical loadability, chemical stability or biocompatibility for all applications and, therefore, adhesion cannot be fully prevented.

The foreign-body reaction to an implant can be minimized or even controlled by the use of steroidal and non-steroidal anti-inflammatory drugs. Glucocorticoids have been used, for example.

Over the long term, most electrode leads adhere to the points at which they rest against the wall of the blood vessel, and therefore cannot be explanted, or only explanted with great difficulty. Many of the above-mentioned materials are suitable only for a limited period of time and cannot permanently prevent the foreign-body reaction and, therefore, adhesion of implants, in particular electrode leads, sensors (connected to leads, or leadless) or leadless pacers. So far, no coatings—either pure polymers or drug-eluting polymers—have been known that solve the problem over periods of time longer than several months.

Explantation of electrode leads in the vicinity of the heart, in particular, is associated with a high risk that important blood vessels or the heart will be injured, in particular, if the electrode leads have adhered, even when specially developed systems are used and the operating surgeon is highly experienced. For this reason, they often remain in the body when they should be replaced.

Although the risks associated with an electrode lead remaining in the body are difficult to assess, they are tolerated at the moment in order to avoid the considerably higher risks associated with explantation, especially in the vicinity of the heart.

The present invention is directed toward overcoming one or more of the above-identified problems. A problem addressed by the present invention is that of providing a medical implant that is more suitable with regard to potential explantation.

### SUMMARY

A problem is solved by an implant having the features of the independent claim(s). Advantageous developments of the inventive idea are the subject matter of the dependent claims. Moreover, a medical arrangement having the features of claim 14 is provided.

The present invention is based on the premise of avoiding the previously common application of high tensile forces for the explantation of medical implants according to the initially stated definition for removal from the tissue environment into which they have adhered. It is further based on the premise, instead, of causing the implant to vibrate, e.g., to more or less “shake”, in order to release it from the tissue environment. Finally, the present invention incorporates the idea of not generating the mechanical vibrations outside of the body and transferring them to the implant by way of a mechanical transmission element but, rather, to generate them directly in the implant by way of suitable energy conversion. For this purpose, a transducer element is provided in the implant, which induces mechanical vibration of the implant when electrically and/or magnetically controlled, to thereby bring about a release from the tissue environment or at least a loosening therefrom. It should be pointed out that this release or loosening does not necessarily have to take place only for the purpose of and at the moment of explantation but, rather, could also be carried out for the purpose of prevention, to prevent fixed adhesion, e.g., at greater intervals during the service life of a long-term implant.

A transducer element in the tip of an electrode or sensor lead, or in a leadless pacer/sensor or a similar implant, will greatly simplify the development of future implant coatings, since it thereby becomes less necessary to keep endogenous cells and adhesions away. A low grade of adhesion can be tolerated because the present invention makes explantation possible despite adhesion in and to bodily tissue.

In addition, drug-eluting coatings which can harm the body are avoided. It is possible to control the point of time at which the implant should be released by the tissue upon explantation.

A further advantage is provided since the endogenous adhesions additionally stabilize the position of the implant. This is of particular significance for lead-connected or leadless sensors located in the pulmonary artery or the vena cava before the heart. If the sensors would slip, serious complications could result.

In one embodiment of the present invention, the implant comprises a transducer element that couples inductively to an alternating magnetic field. In an embodiment that is preferred from a current perspective, however, it is an electrically controllable transducer element, such as, for example, a piezoceramic vibration element.

In a further embodiment, the transducer element is in the form of a separate transducer element inserted into the implant. In another embodiment, the transducer element is disposed in the wall region, in particular, being embedded in the wall over a large surface area. If the latter embodiment is combined with the embodiment as an electrically controllable piezo element, an advantageous embodiment results in which the transducer element comprises a piezoceramic foil or a piezoelectric polymer foil.

The above-mentioned piezo foils are a few μm thick and do not substantially increase the diameter of an implant. They can be produced in a tube shape, for example, which results in a few advantages in production. It should also be provided that the piezo foils extend along the entire longitudinal axis of the implant, if possible, because it is impossible to determine exactly where an implant will adhere into the tissue.

In a further embodiment of the present invention, the proposed implant comprises an electrical connection for contacting the transducer element by way of a temporary control line serving as an explantation tool. In another embodiment, an integrated control line is provided for connection to an internal control device, a control device disposed in a further implant, or an extracorporeal control device.

In yet another embodiment, the transducer element is designed, or additional energy-supply means are provided, such that wireless control can take place by way of an extracorporeally generated, alternating magnetic field (generated by an excitation coil held at the body in the vicinity of the implant, for example). An advantage of the wireless energy supply of the transducer element would be that no additional technology would be required in the main implant, and no additional electrode leads would be required.

In an application of the present invention that is particularly important from a current perspective, the implant is designed as an implantable electrode lead or sensor lead. In a further embodiment that is interesting with regard to perspective, the implant is a leadless cardiac stimulation device or cardioversion device, or a lead-connected or leadless sensor for intracorporeal, physical, physiological or biochemical measured quantities, which is known per se having a cylindrical basic shape. In both application forms, a piezoceramic foil or a piezoelectric polymer foil, in particular, in sleeve form or annular segment form, can be disposed, advantageously, in or on the wall of a distal section.

The proposed medical arrangement which comprises an implant of the above-described type furthermore comprises a control device for the electrical and/or magnetic control of the transducer element and coupling means for coupling energy therein. It can be an arrangement (comprising, for example, a pacemaker electrode lead and a specially equipped cardiac pacemaker) which is implantable in its entirety. From a current perspective, however, an arrangement in which the control device is in the form of an extracorporeal explantation support device is clinically more interesting.

Further features, aspects, objects, advantages, and possible applications of the present invention will become apparent from a study of the exemplary embodiments and examples described below, in combination with the figures, and the appended claims.

### DESCRIPTION OF THE DRAWINGS

Advantages and useful features of the present invention will also become apparent from the basic description that follows of exemplary embodiments, with reference to the figures. In the figures:

FIG. 1A shows a schematic diagram of a first embodiment of the implant according to the present invention, using the example of a section of the electrode body of an electrode lead,

FIG. 1B shows a schematic diagram of a further embodiment of the implant according to the present invention, using the example of an electrode lead,

FIG. 2 shows a schematic diagram of a further embodiment of the implant according to the present invention, using the example of an electrode lead,

FIG. 3 shows a schematic diagram of a further embodiment of the implant according to the present invention, using the example of an electrode lead,

FIG. 4 shows a schematic diagram of a further embodiment of the implant according to the present invention, using the example of a leadless pacemaker,

FIG. 5 shows a schematic depiction of a first embodiment of the medical device according to the present invention, and

FIG. 6 shows a schematic depiction of a further embodiment of the proposed medical arrangement.

### DETAILED DESCRIPTION

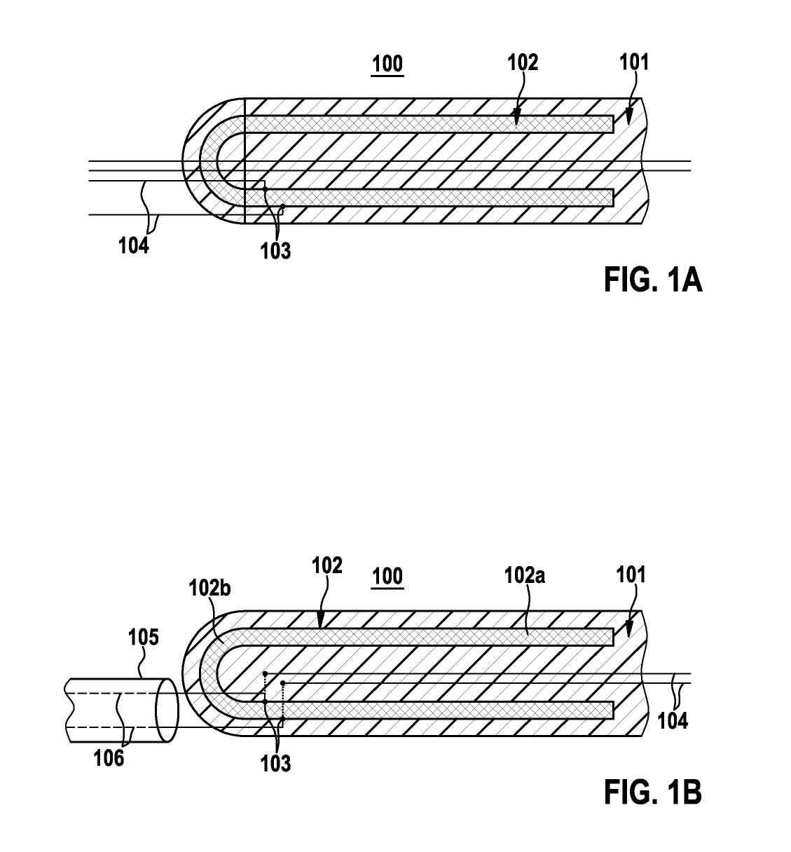

FIGS. 1A-1B show, schematically, only the parts of the distal end of an electrode lead 100 that are essential in conjunction with the present invention, which otherwise comprises—in a manner known per se—one or more electrode poles for stimulating excitable bodily tissue and/or for sensing tissue potentials and, optionally, one or more sensors for the detection of further physiological variables in the body of a patient. Those parts and the supply leads and terminal leads thereof are not depicted, and are not described further here since they are familiar and well-knows and understood to a person skilled in the art. Furthermore, the features described specifically with reference to an electrode lead can also be used in the other devices referred to by the term “implant”.

FIG. 1A shows a longitudinal cross section of an electrode body section of an electrode lead 100. A piezoceramic foil 102, which comprises an elongated sleeve in the embodiment shown here, is embedded in a plastic jacket 101 of the electrode lead 100. The piezoceramic foil 102 extends across at least one section of the electrode body, which extends between the distal end pointing toward the treatment site and a proximal end in the direction of the implanted electromedical device. Furthermore, supply leads 104 for the electrical connection of the therapeutic electrodes or sensor electrodes on the distal end of the electrode lead extend in the electrode body. By way of connector contacts 103 on the inner and outer foil surfaces, the piezoceramic foil 102 is connected to electric supply leads 104 in the interior of the electrode body, which, in turn, are connected to an electromedical device which is likewise implanted, or to an external device, especially in the case of an explantation. As depicted in the following embodiment according to FIG. 1B, the piezoceramic foil 102 can also be contacted externally by way of a separate cable 105, which is guided thereto for explantation, to supply leads 106 provided therein.

If an alternating voltage having a suitable frequency and voltage is applied to the piezoceramic foil 102, it radiates acoustic waves. The radiated acoustic waves induce the separation or loosening of the adhesion from the surface of the electrode. The frequency of the impressed alternating voltage can be, for example, in the range of 20 kHz to 20 MHz, and preferably, a frequency between 50 kHz and 100 kHz is used. The alternating voltage can be impressed as a continuous alternating voltage having an arbitrary curve shape, or in the form of bursts or pulses.

In a variant of the embodiment depicted, the transducer element can be designed with annular segments which are electrically interconnected, and it can be made of a piezoelectric material, e.g., lead zirconate titante (PZT), barium titanate or lithium niobate. The flexibility of the electrode in this region is increased by way of the annular segments. Alternatively to the piezoceramic foil, it is also possible to use a foil made of a piezoelectric polymer (e.g., PVDF; polyvinylidene fluoride).

FIG. 1B shows a further embodiment of an electrode lead 100. Depicted is the distal end of the electrode lead 100. A piezoceramic foil 102 is embedded in a plastic jacket 101 of the electrode lead 100, which, in the embodiment shown here, comprises an elongated sleeve 102a and a hemispherical cap 102b in the region of the distal electrode tip (which is likewise hemispherical). By way of connection contacts 103 on the inner and outer foil surfaces, the piezoceramic foil 102 is connected either (by way of dotted lines in the case) to electric supply leads 105 in the interior of the electrode body, which, in turn, are electrically connected to a likewise implanted, electromedical device or—especially in the case of an explantation—to an external device by way of an electrode plug which is present on the proximal end of the electrode lead 100 and is not depicted, or it can be contacted externally to supply leads 106 provided therein by way of a separate cable 105 guided thereto for the explantation. For better illustration of the principle, the cable 105 is guided schematically from the distal side to the piezoceramic foil 102. Of course, a person skilled in the art understands that, in the case of explantation, this cable is introduced into the interior of the electrode body from the proximal direction.

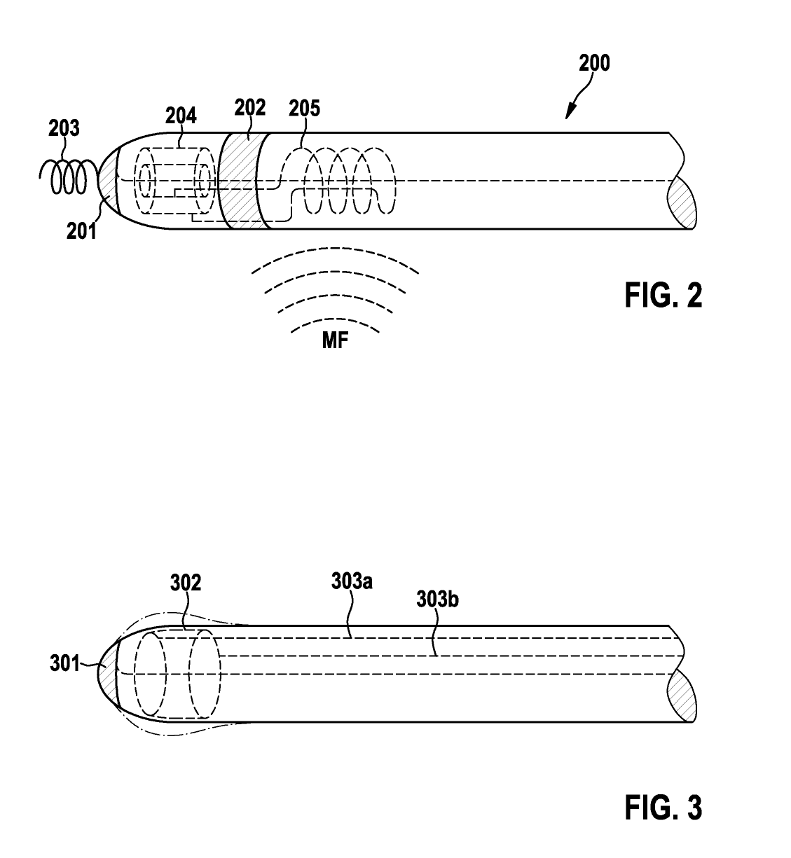

FIG. 2 shows, as a sketch of a variant of the embodiment depicted in FIG. 1B, the distal end of an electrode lead 200 comprising a tip electrode 201 and a ring electrode 202, on the distal end of which a fixing coil 203 is provided for anchoring in the bodily tissue to be stimulated, such as, for example, in the trabeculae carnea of the heart. A piezoceramic vibrating body 204 in the form of a hollow cylinder is installed in the electrode lead 200, near the distal end, as the transducer element, the inner and outer walls of which are connected to the ends of a receive coil 205.

By way of this coil 205, the energy for generating the acoustic wave is supplied wirelessly using magnetic-inductive coupling. In explantation, this takes place by way of a suitable (not depicted) transmit coil which is held at the body on the outside. This solution has the advantage that the electrode does not require any additional connectors and no additional special devices are required in the IMD for generating and supplying the alternating voltage. This electrode is therefore fully compatible with conventional electrode connectors and IMDs.

FIG. 3 shows, as a further embodiment, an electrode lead 300 which comprises a tip electrode 301, as the only electrode, and the end of which—symbolized by the dashed bulge of the distal end—is elastically compressible to prevent penetration of bodily walls if wall contact occurs. A capacitive pressure sensor 302 (with a compressible conductive foam, for example) is provided close to the distal end to determine a compressive force if wall contact by the electrode tip occurs. It is connected at the distal and proximal end faces thereof by way of an electrode and a supply lead 303a, 303b connected there to a (not depicted) proximal connection contact of the electrode lead.

A further possibility for generating the required acoustic waves is therefore utilized. Capacitive pressure sensors can be used to generate acoustic waves by applying an alternating voltage to the pressure-measuring capacitor. They then function (quasi inversely) as CMUTs (capacitive micromachined ultrasonic transducers). The device for generating the alternating voltage can be included, for example, in the devices for determining the pressure signal on the basis of the capacitance of the capacitive pressure sensor. Alternatively, this alternating voltage can likewise be coupled wirelessly magnetically-inductively by way of a suitable coil.

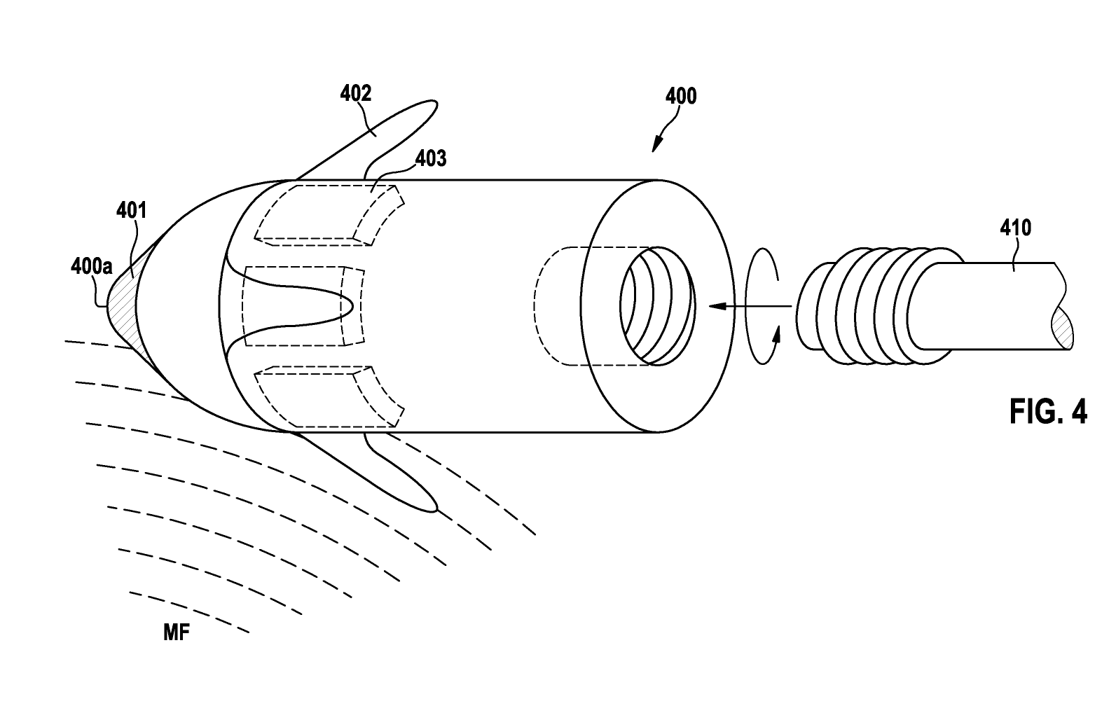

FIG. 4 shows, as a further embodiment of the present invention, a leadless pacemaker 400, the basic shape of which is cylindrical, and one end of which terminates in a rounded tip 400a, at which a stimulation electrode 401 is disposed. Plastic fins 402 close to this end of the pacemaker 400 are provided for anchoring in branched bodily tissues at the application site of the pacemaker. In addition to the usual components of such a device, the pacemaker 400 comprises a ring of oscillating bodies 403, which can be excited inductively by way of an external alternating magnetic field (MF) to vibrate, and which are placed near the attachment point of the fins 402. The oscillating bodies 403 excite the fins 402, in particular, to undergo elastic oscillations which loosen the anchoring thereof in the branched bodily tissue, and thereby create the preconditions for explantation of the pacemaker 400 using an explantation tool 410 (which is depicted here merely symbolically as a guide wire having a terminal outer thread). The explantation tool 410 according to this embodiment can also serve as a further embodiment of the contact possibility to the aforementioned oscillating bodies 403, mentioned in reference to FIGS. 1 and 2. In this case, the electric energy is achieved by way of galvanic contacting between the explantation tool—in particular, by way of electrically conductive contacts at the surface of the explantation tool—and oscillating bodies. Contact surfaces in the interior of the leadless pacemaker 100 ensure this contact. Of course, this type of coupling of energy is also possible with the others included in the term “implant” as defined herein.

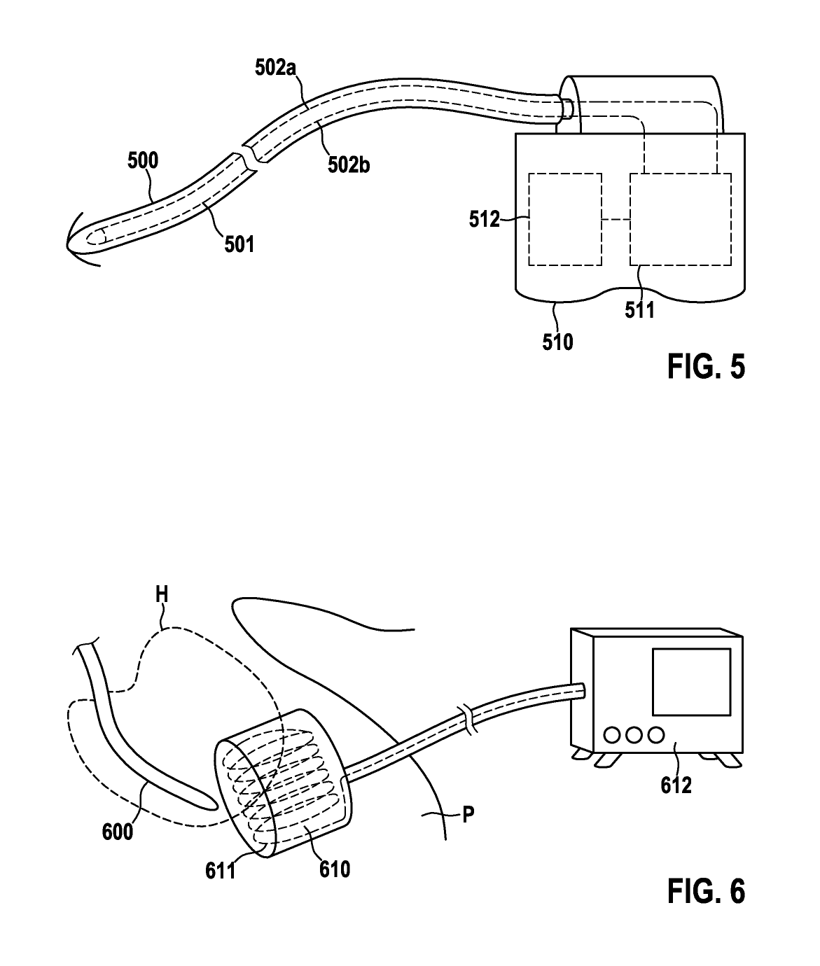

FIG. 5 shows schematically, as a first example of a medical arrangement according to the present invention, an electrode lead 500 comprising piezofoil 501 embedded close to the distal end thereof, and two electric supply leads 502a, 502b therefore, which is connected to the cardiac pacemaker 510 such that the leads 502a, 502b in the pacemaker are connected to an explantation transducer generator 511. It is activated to prepare for explantation of the lead 500, and is supplied with energy for a predetermined period of time by way of the pacemaker battery 512, in order to induce vibrations to loosen the lead end from the surrounding cardiac tissue.

As an alternative embodiment, FIG. 6 shows the distal end of an electrode lead 600 placed in the heart H of a patient P, which is equipped, in the manner of the embodiment depicted in FIG. 2, with a piezoceramic in connection with a receive coil or, also in the manner of the “leadless pacer” depicted in FIG. 4 to an inductively driven oscillating body. To prepare for explantation of this electrode lead, an energy supply head 610 is guided from the outside to the applicable bodily region, which contains a transmit coil 611 and is connected to an external supply and control device 612. The energy supply from the energy supply head 610 into the transducer element (not depicted separately) in the electrode lead 600 takes place in the manner described above using an electromagnetic alternating field. The alternating voltage therefore is provided by a generator contained in the supply and control device 612 for a suitable time period which is sufficient for loosening the electrode lead and is not harmful to the health of the patient.

The embodiments of the present invention are not limited to the above-described examples and emphasized aspects but, rather, are possible in a large number of modifications that lie within the scope of handling by a person skilled in the art.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/We claim:

1. A medical implant comprising:
a transducer element which induces mechanical vibrations of the implant when electrically and/or magnetically controlled.

2. The implant according to claim 1, wherein the transducer element comprises a transducer element coupling inductively to an alternating magnetic field.

3. The implant according to claim 1, wherein the transducer element comprises an electrically controllable transducer element.

4. The implant according to claim 1, wherein the transducer element is in the form of an oscillating body inserted separately into the implant.

5. The implant according to claim 1, wherein the transducer element is disposed in a wall region of the implant and embedded in the wall region over a large surface area.

6. The implant according to claim 3, wherein the transducer element comprises a piezoceramic foil or a piezoelectric polymer foil.

7. The implant according to claim 1, further comprising an electrical connection for the contacting of the transducer element using a temporary control line serving as an explantation aid.

8. The implant according to claim 1, further comprising an integrated control line for connection to an internal control device, a control device disposed in a further implant, or an extracorporeal control device.

9. The implant according to claim 1, wherein the transducer element is designed, or additional energy supply means are provided, for wireless control by way of an extracorporeally generated electromagnetic alternating field.

10. The implant according to claim 9, wherein the implant further comprises a receive coil device which is inserted into the transducer element or is connected thereto.

11. The implant according to claim 1, which is in the form of an implantable electrode lead or sensor lead.

12. The implant according to claim 11, wherein a piezoceramic foil or a piezoelectric polymer foil, in sleeve form or annular segment form, is disposed in or on the wall of a distal section.

13. The implant according to claim 1, which is in the form of a leadless cardiac stimulation or cardioversion device or as a leadless sensor.

14. The implant according to claim 13, wherein a piezoceramic foil or a piezoelectric polymer foil, in sleeve form or annular segment form, is disposed in or on the wall of a distal section.

15. A medical arrangement comprising:
an implant according to claim 1; and
a control device for the electrical and/or magnetic control of the transducer element and coupling means for coupling energy therein.

16. The arrangement according to claim 15, wherein the control device is in the form of an extracorporeal explantation support device.
